(19) United States
(12) Patent Application Publication
(10) Pub. No.: US 2023/0302010 A1
(43) Pub. Date: Sep. 28, 2023

(54) 2-((4S)-6-(4-CHLOROPHENYL)-1-METHYL-4H-BENZO[C]ISOXAZOLO[4,5-E]AZEPIN-4-YL)ACETAMIDE FOR TREATING THROMBOCYTHEMIA

(71) Applicant: Constellation Pharmaceuticals, Inc., Boston, MA (US)

(72) Inventor: Adrian Senderowicz, Somerville, MA (US)

(21) Appl. No.: 18/019,634

(22) PCT Filed: Aug. 3, 2021
(86) PCT No.: PCT/US2021/044318

§ 371 Date: Feb. 3, 2023

Related U.S. Application Data
(60) Provisional application No. 63/060,723, filed on Aug. 4, 2020.

(51) Int. Cl. A61K 31/55; A61P 7/02

(52) U.S. Cl. CPC A61K 31/55; A61P 7/02

(57) ABSTRACT

The present disclosure relates to the use of 2-((4S)-6-(4-chlorophenyl)-1-methyl-4H-benzo[c]isoxazolo[4,5-e]azepin-4-yl)acetamide, and pharmaceutically acceptable salts thereof, for treating thrombocythemia.

### RELATED APPLICATIONS

This application claims the benefit of priority to U.S. Provisional Application No. 63/060,723, filed Aug. 4, 2021, the entire contents of which are incorporated herein by reference.

### BACKGROUND

Essential thrombocythemia (ET) is one of the three Philadelphia chromosome-negative myeloproliferative neoplasms (MPNs) and it is characterized by excessive clonal platelet production. About 90% of patients with ET have somatically acquired mutations in JAK2V617F, calreticulin (CALR), or thrombopoietin receptor (MPL). Although the roles of JAK2V617F, CALR, and MPL mutations in disease phenotype, leukemic transformation, and stem cell involvement are uncertain, MPN gene expression signatures are related to the upregulation of JAK/STAT signaling.

ET affects approximately 38 to 50 patients per every 100,000 patients in the United States and is classified as a rare disease by the National Institutes of Health. Epidemiological studies estimate that the incidence of this disease ranges from about 1 to 2.5 new cases/100,000 per year. The median age at diagnosis of ET is 65 to 70 years, and 20% of patients with ET are younger than 40 years old. The male-to-female patient ratio is approximately 1:2.

Patients with ET have multiple quality of life- and survival-diminishing complications, including life-threatening thrombotic arterial and venous events, making it a serious disease. Up to half of all patients with ET experience vasomotor, thrombotic, or hemorrhagic events over the course of their disease. Vasomotor symptoms, including headache, lightheadedness, syncope, atypical chest pain, acral paresthesia, livedo reticularis, erythromelalgia, seizures, psychiatric deficits, and visual disturbances, afflict 13% to 40% of patients with ET. Pruritus occurs in 5% of patients and other symptoms seen in patients include enlarged lymph nodes, digital ulcers, dysphoria, and others.

The chronic nature of ET necessitates a well-tolerated, long-term treatment, with a primary goal of lowering platelet counts to minimize the risk of thrombotic or hemorrhagic events while managing toxicity and symptoms. For patients with low-risk ET, low-dose aspirin and observation are sufficient. For patients with high-risk ET, physicians provide low-dose aspirin in combination with cytoreductive therapy. Although not approved for the treatment of ET in the US, hydroxyurea (HU) serves as standard of care for high-risk ET and is recommended as first-line cytoreductive therapy for these patients. HU is generally well-tolerated and offers important clinical benefits, but it also carries major risks, including development of anemia, cutaneous complications, and leukemic transformation. In addition, approximately 20% of patients who take HU become refractory and/or intolerant to the drug, as defined by European Leukemia Net (ELN). See Harrison et al., Blood. 2017; 130(17):1889-1897 and Barosi et al Leukemia. 2007; 21(2):277-280. Life expectancy for patients with HU-resistance/intolerance is shorter than for patients who respond to HU. See Barosi et al., Blood. 2009; 113(20):4829-4833. In a 14-year retrospective study involving 166 patients with high-risk ET treated with HU for a median of 4.5 years, 38 (23%) patients died at a median follow-up of seven years from ET diagnosis, resulting in a 65% survival from the beginning of HU treatment. See Hernandez-Boluda et al., Br J Haematol. 2011; 152(1):81-88. Patients who achieved a complete response (platelet count ≤400×109/L, white blood cell (WBC) count ≤10×109/L, normal spleen size, and no disease-related symptoms) to HU had a 79% 10-year overall survival; comparatively, those with HU resistance had a meager 26% 10-year overall survival. Patients with high-risk ET who are resistant and/or intolerant to HU also have a greater risk of fibrotic and leukemic disease transformation than patients who respond to HU. See Hernandez-Boluda et al., Br J Haematol. 2011; 152(1):81-88.

Given the barren treatment landscape for patients with high-risk ET who are ineligible, intolerant, and/or resistant to HU, high-risk ET patients must alternate between treatments and ultimately resort to experimental therapies in clinical trials. Furthermore, none of the current treatments available exhibit disease-modifying properties or alleviate symptom burden. See Mora et al., Expert Rev Hematol. 2019; 12(3):159-171. This warrants the need to create new therapies for patients with high-risk ET.

### SUMMARY

Provided herein are methods of using 2-((4S)-6-(4-chlorophenyl)-1-methyl-4H-benzo[c]isoxazolo[4,5-e]azepin-4-yl)acetamide, an inhibitor of the Bromodomain and Extra-Terminal (BET) family and referenced herein as Compound 1, and pharmaceutically acceptable salts thereof, for treating thrombocythemia (e.g., ET or high-risk ET). Compound 1 was shown to inhibit aberrant megakaryocyte differentiation and proliferation. See the Exemplification section below. Aberrant megakaryocytes are drivers of inflammation and altered hematopoiesis in ET.

In one aspect, the methods described herein are useful for subjects who are resistant or intolerant to treatment with hydroxyurea (HU) or HU related therapies.

In one aspect, treatment with Compound 1 is intended to normalize platelet levels in subjects having thrombocythemia (e.g., ET or high-risk ET).

In one aspect, treatment with Compound 1 is also intended to increase hemoglobin levels in subjects having thrombocythemia (e.g., ET or high-risk ET). This result is useful in subjects who are also anemic or have become anemic as a result of having thrombocythemia. Long term use of HU for the treatment of ET has led to anemia. See Briere Orphanet J Rare Dis. 2007; 2: 3.

In one aspect, treatment with Compound 1 is further intended to improve white blood cell counts in a subject having thrombocythemia (e.g., essential or high-risk essential thrombocythemia). White blood cell counts are typically elevated in subject who have ET. See e.g., the National Organization of Rare Disorders database and Barbui et al., Blood. 2009 Jul. 23; 114(4): 759-763. In addition, long term use of HU for the treatment of ET has led to neutropenia. See Briere Orphanet J Rare Dis. 2007; 2: 3.

In one aspect, treatment with Compound 1 is further intended to reduce spleen size in subjects having thrombocythemia (e.g., essential or high-risk essential thrombocythemia). Spleen enlargement is present in 10-20% of ET patients at diagnosis. See Alessandro Andriani et al., Am J Hematol, 2016 Mar.;91(3):318-21.

### BRIEF DESCRIPTION OF THE FIGURES

FIG. 1 shows the effects of Compound 1 on IL6 and IL10 mRNA transcript levels.

FIG. 2 depicts histograms of Compound 1 effect on megakaryocyte differentiation.

### DETAILED DESCRIPTION

In a first aspect, provided are methods of treating essential thrombocythemia (ET) in a subject in need thereof, comprising administering to the subject a therapeutically effective amount of 2-((4S)-6-(4-chlorophenyl)-1-methyl-4H-benzo[c]isoxazolo[4,5-e]azepin-4-yl)acetamide, or a pharmaceutically acceptable salt thereof. Alternatively, as part of a first aspect, provided is a therapeutically effective amount of 2-((4S)-6-(4-chlorophenyl)-1-methyl-4H-benzo[c]isoxazolo[4,5-e]azepin-4-yl)acetamide, or a pharmaceutically acceptable salt thereof, for use in treating ET. In another alternative, further provided as part of a first aspect, is the use of a therapeutically effective amount of 2-((4S)-6-(4-chlorophenyl)-1-methyl-4H-benzo[c]isoxazolo[4,5-e]azepin-4-yl)acetamide, or a pharmaceutically acceptable salt thereof, for the manufacture of a medicament for treating ET.

2-((4S)-6-(4-chlorophenyl)-1-methyl-4H-benzo[c]isoxazolo[4,5-e]azepin-4-yl)acetamide, also referred to herein as Compound 1, is exemplified as Compound 144 in U.S. Pat. No. 8,796,261, and is represented by the following structural formula:

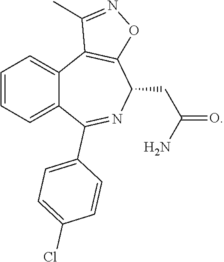 [SMILES: Cc1noc2c1-c1ccccc1C(c1ccc(Cl)cc1)=N[C@H]2CC(N)=O]

The entire contents of U.S. Pat. No. 8,796,261 are incorporated by reference herein.

Thrombocythemia is a known condition in which there is an excess number of platelets in the blood. Too many platelets can lead to certain conditions, including stroke, heart attack, or a clot in the blood vessels. There are two types of thrombocythemia: primary and secondary. Primary thrombocythemia, synonymous with essential thrombocythemia (ET), is a disease in which abnormal cells in the bone marrow cause an increase in platelets. Subjects with ET have a later risk of developing myelofibrosis (MF). MF is distinct from ET in that bone marrow function has been impacted by scarring in subjects who have MF. Thus, the manufacture platelets via bone marrow is compromised. With ET, there is an innate problem of hematopoiesis in the bone marrow. Secondary thrombocythemia is caused by another condition such as anemia, cancer, inflammation, infection, or as a result of surgery (e.g., splenectomy).

In one aspect, subjects to be treated by the methods described herein are said to have ET if they are diagnosed according to the revised 2016 World Health Organization (WHO) guidelines (Blood Cancer J. 2018 February; 8(2): 15)). For example, according to the WHO, all four of the following major criteria or the first three major criteria and the following minor criterion must be met: Major criteria=1) a platelet count of greater than or equal to 450×10⁹/L (≥450,000 μL); 2) a bone marrow biopsy showing proliferation mainly of the megakaryocyte lineage with increased numbers of enlarged, mature megakaryocytes with hyperlobulated nuclei. No significant increase or left shift in neutrophil granulopoiesis or erythropoiesis and very rarely minor (grade 1) increase in reticulin fibers; 3) not meeting WHO criteria for BCR-ABL1+CML, PV, PMF, MDS, or other myeloid neoplasms [see e.g., Blood (2016) 127 (20): 2391-2405]; and 4) the presence of JAK2, CALR or MPL mutation. Minor criteria=demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia).

Patients with ET may be categorized into very low, low, medium, and high risk, as defined by the revised International Prognostic Score of Thrombosis for Essential Thrombocythemia (IPSET). See Barbui et al., Leukemia. 2018; 32(5):1057-1069 and Heidar et al., Am J Hematol. 2016; 91(4):390-394. Because a high platelet count is associated with thrombosis and/or hemorrhage, the patient prognosis criteria focuses on risk of thrombotic or hemorrhagic complications. See Tefferi et al., Mayo Clin Proc. 2015; 90(9):1283-1293. These four levels of risk are determined from four adverse variables: thrombosis history, age >60 years, cardiovascular (CV) risk factors, and presence of JAK2V617F mutation. In one aspect, subjects to be treated by the methods described herein are considered to be high-risk if they have a history of thrombosis or are greater than 60 years with JAK2 mutations; CV risk factors do not have a strong predictive effect specifically on patients with high-risk whose disease status is determined by old age or history of thrombosis.

Patients with ET may also be categorized as high risk using criteria set forth in the MAJIC-ET study by Harrison et al., Blood. 2017; 130(17):1889-1897. Therefore, in another aspect, subjects to be treated by the methods described herein are considered to be high-risk if they further possess any one of the following characteristics: 1) are age 60 years or older; 2) have a platelet count of greater than 1500×10⁹/L (at any point during the patient's disease); 3) have a previous documented thrombosis (including transient ischemic attack (TIA)), erythromelalgia or migraine (severe, recurrent, requiring medications, and felt to be secondary to the MPN) either after diagnosis or within 10 years before diagnosis and considered to be disease-related; 4) have a previous hemorrhage related to ET; 5) have diabetes or hypertension requiring pharmacological therapy for greater than 60 months.

In a first aspect, a subject to be treated by the methods described herein has ET characterized by the subject 1) having a platelet count of greater than or equal to 450×10⁹/L (≥450,000 μL); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) the presence of JAK2, CALR or MPL mutation.

Alternatively, as part of a first aspect, a subject to be treated by the methods described herein has ET characterized by the subject 1) having a platelet count of greater than or equal to 500×10⁹/L, greater than or equal to 550×10⁹/L, greater than or equal to 600×10⁹/L, greater than or equal to 650×10⁹/L, greater than or equal to 700×10⁹/L, greater than or equal to 750×10⁹/L, greater than or equal to 800×10⁹/L, greater than or equal to 850×10⁹/L, greater than or equal to 900×10⁹/L, greater than or equal to 950×10⁹/L, greater than or equal to 1,000×10⁹/L, greater than or equal to 1,050×10⁹/L, greater than or equal to 1,100×10⁹/L, greater than or equal to 1,150×10⁹/L, greater than or equal to 1,200×10⁹/L, greater than or equal to 1,250×10⁹/L, greater than or equal to 1,300×10⁹/L, greater than or equal to 1,350×10⁹/L, greater than or equal to 1,400×10⁹/L, or greater than or equal to 1,450×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) the presence of JAK2, CALR or MPL mutation.

Alternatively, as part of a first aspect, a subject to be treated by the methods described herein has ET characterized by the subject 1) having a platelet count ranging from 450×10⁹/L to 1500×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) the presence of JAK2, CALR or MPL mutation.

Alternatively, as part of a first aspect, a subject to be treated by the methods described herein has ET characterized by the subject 1) having a platelet count ranging from 450×10⁹/L to 1500×10⁹/L, 500×10⁹/L to 1500×10⁹/L, 550×10⁹/L to 1500×10⁹/L, 600×10⁹/L to 1500×10⁹/L, 650×10⁹/L to 1500×10⁹/L, 700×10⁹/L to 1500×10⁹/L, 750×10⁹/L to 1500×10⁹/L, 800×10⁹/L to 1500×10⁹/L, 850×10⁹/L to 1500×10⁹/L, 900×10⁹/L to 1500×10⁹/L, 950×10⁹/L to 1500×10⁹/L, 1,000×10⁹/L to 1500×10⁹/L, 1,050×10⁹/L to 1500×10⁹/L, 1,100×10⁹/L to 1500×10⁹/L, 1,150×10⁹/L to 1500×10⁹/L, 1,200×10⁹/L to 1500×10⁹/L, 1,250×10⁹/L to 1500×10⁹/L, 1,300×10⁹/L to 1500×10⁹/L, 1,350×10⁹/L to 1500×10⁹/L, 1,400×10⁹/L to 1500×10⁹/L, or 1,450×10⁹/L to 1500×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) the presence of JAK2, CALR or MPL mutation.

Alternatively, as part of a first aspect, a subject to be treated by the methods described herein has ET characterized by the subject having a platelet count ranging from 450×10⁹/L to 1450×10⁹/L, 450×10⁹/L to 1400×10⁹/L, 450×10⁹/L to 1350×10⁹/L, 450×10⁹/L to 1300×10⁹/L, 450×10⁹/L to 1250×10⁹/L, 450×10⁹/L to 1200×10⁹/L, 450×10⁹/L to 1150×10⁹/L, 450×10⁹/L to 1100×10⁹/L, 450×10⁹/L to 1050×10⁹/L, 450×10⁹/L to 1000×10⁹/L, 450×10⁹/L to 950×10⁹/L, 450×10⁹/L to 900×10⁹/L, 450×10⁹/L to 950×10⁹/L, 450×10⁹/L to 900×10⁹/L, 450×10⁹/L to 850×10⁹/L, 450×10⁹/L to 800×10⁹/L, 450×10⁹/L to 750×10⁹/L, 450×10⁹/L to 700×10⁹/L, 450×10⁹/L to 650×10⁹/L, 450×10⁹/L to 600×10⁹/L, 450×10⁹/L to 550×10⁹/L, or 450×10⁹/L to 500×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) the presence of JAK2, CALR or MPL mutation.

In a second aspect, a subject to be treated by the methods described herein has ET characterized by the subject 1) having a platelet count of greater than or equal to 450×10⁹/L (≥450,000 μL); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) demonstration of another clonal marker (e.g., ASXL1, EZH2, TET2, IDH1/IDH2, SRSF2, or SR3B1 mutation) or no identifiable cause of thrombocytosis (e.g., infection, inflammation, iron deficiency anemia).

Alternatively, as part of a second aspect, a subject to be treated by the methods described herein has ET characterized by the subject 1) having a platelet count of greater than or equal to 500×10⁹/L, greater than or equal to 550×10⁹/L, greater than or equal to 600×10⁹/L, greater than or equal to 650×10⁹/L, greater than or equal to 700×10⁹/L, greater than or equal to 750×10⁹/L, greater than or equal to 800×10⁹/L, greater than or equal to 850×10⁹/L, greater than or equal to 900×10⁹/L, greater than or equal to 950×10⁹/L, greater than or equal to 1,000×10⁹/L, greater than or equal to 1,050×10⁹/L, greater than or equal to 1,100×10⁹/L, greater than or equal to 1,150×10⁹/L, greater than or equal to 1,200×10⁹/L, greater than or equal to 1,250×10⁹/L, greater than or equal to 1,300×10⁹/L, greater than or equal to 1,350×10⁹/L, greater than or equal to 1,400×10⁹/L, or greater than or equal to 1,450×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) demonstration of another clonal marker (e.g., ASXL1, EZH2, TET2, IDH1/IDH2, SRSF2, or SR3B1 mutation) or no identifiable cause of thrombocytosis (e.g., infection, inflammation, iron deficiency anemia).

Alternatively, as part of a second aspect, a subject to be treated by the methods described herein has ET characterized by the subject 1) having a platelet count ranging from 450×10⁹/L to 1500×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) demonstration of another clonal marker (e.g., ASXL1, EZH2, TET2, IDH1/IDH2, SRSF2, or SR3B1 mutation) or no identifiable cause of thrombocytosis (e.g., infection, inflammation, iron deficiency anemia).

Alternatively, as part of a second aspect, a subject to be treated by the methods described herein has ET characterized by the subject 1) having a platelet count ranging from 450×10⁹/L to 1500×10⁹/L, 500×10⁹/L to 1500×10⁹/L, 550×10⁹/L to 1500×10⁹/L, 600×10⁹/L to 1500×10⁹/L, 650×10⁹/L to 1500×10⁹/L, 700×10⁹/L to 1500×10⁹/L, 750×10⁹/L to 1500×10⁹/L, 800×10⁹/L to 1500×10⁹/L, 850×10⁹/L to 1500×10⁹/L, 900×10⁹/L to 1500×10⁹/L, 950×10⁹/L to 1500×10⁹/L, 1,000×10⁹/L to 1500×10⁹/L, 1,050×10⁹/L to 1500×10⁹/L, 1,100×10⁹/L to 1500×10⁹/L, 1,150×10⁹/L to 1500×10⁹/L, 1,200×10⁹/L to 1500×10⁹/L, 1,250×10⁹/L to 1500×10⁹/L, 1,300×10⁹/L to 1500×10⁹/L, 1,350×10⁹/L to 1500×10⁹/L, 1,400×10⁹/L to 1500×10⁹/L, or 1,450×10⁹/L to 1500×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) demonstration of another clonal marker (e.g., ASXL1, EZH2, TET2, IDH1/IDH2, SRSF2, or SR3B1 mutation) or no identifiable cause of thrombocytosis (e.g., infection, inflammation, iron deficiency anemia).

Alternatively, as part of a second aspect, a subject to be treated by the methods described herein has ET characterized by the subject 1) having a platelet count ranging from 450×10⁹/L to 1450×10⁹/L, 450×10⁹/L to 1400×10⁹/L, 450×10⁹/L to 1350×10⁹/L, 450×10⁹/L to 1300×10⁹/L, 450×10⁹/L to 1250×10⁹/L, 450×10⁹/L to 1200×10⁹/L, 450×10⁹/L to 1150×10⁹/L, 450×10⁹/L to 1100×10⁹/L, 450×10⁹/L to 1050×10⁹/L, 450×10⁹/L to 1000×10⁹/L, 450×10⁹/L to 950×10⁹/L, 450×10⁹/L to 900×10⁹/L, 450×10⁹/L to 950×10⁹/L, 450×10⁹/L to 900×10⁹/L, 450×10⁹/L to 850×10⁹/L, 450×10⁹/L to 800×10⁹/L, 450×10⁹/L to 750×10⁹/L, 450×10⁹/L to 700×10⁹/L, 450×10⁹/L to 650×10⁹/L, 450×10⁹/L to 600×10⁹/L, 450×10⁹/L to 550×10⁹/L, or 450×10⁹/L to 500×10⁹/L; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; and 4) demonstration of another clonal marker (e.g., ASXL1, EZH2, TET2, IDH1/IDH2, SRSF2, or SR3B1 mutation) or no identifiable cause of thrombocytosis (e.g., infection, inflammation, iron deficiency anemia).

In a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented thrombosis (or undocumented or current thrombosis); iii) a previous hemorrhage related to ET (or current hemorrhage related to ET); and iv) diabetes or hypertension requiring pharmacological therapy for greater than 60 months (past or present).

Alternatively, as part of a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented thrombosis (or undocumented or current thrombosis); iii) a previous hemorrhage related to ET (or current hemorrhage related to ET); and iv) diabetes or hypertension requiring pharmacological therapy for greater than 60 months (past or present).

Alternatively, as part of a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented transient ischaemic attack (TIA) (or undocumented or current TIA); iii) a previous hemorrhage related to ET (or current hemorrhage related to ET); and iv) diabetes or hypertension requiring pharmacological therapy for greater than 60 months (past or present).

Alternatively, as part of a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented transient ischaemic attack (TIA) (or undocumented or current TIA); iii) a previous hemorrhage related to ET (or current hemorrhage related to ET); and iv) diabetes or hypertension requiring pharmacological therapy for greater than 60 months (past or present).

Alternatively, as part of a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented erythromelalgia (or undocumented or currently erythromelalgia); iii) a previous hemorrhage related to ET (or current hemorrhage related to ET); and iv) diabetes or hypertension requiring pharmacological therapy for greater than 60 months (past or present).

Alternatively, as part of a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented erythromelalgia (or undocumented or current erythromelalgia); iii) a previous hemorrhage related to ET (or current hemorrhage related to ET); and iv) diabetes or hypertension requiring pharmacological therapy for greater than 60 months (past or present).

Alternatively, as part of a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented migraine that is severe, recurrent, requiring medication, and believed to be secondary to the ET (or undocumented or current migraine that is severe, recurrent, requiring medication, and believed to be secondary to the ET); iii) a previous hemorrhage related to ET (or current hemorrhage related to ET); and iv) diabetes or hypertension requiring pharmacological therapy for greater than 60 months (past or present).

Alternatively, as part of a third aspect, a subject to be treated by the methods described herein has high-risk ET characterized by the subject 1) having a platelet count as described above in the first or second aspect; 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented migraine that is severe, recurrent, requiring medication, and believed to be secondary to the ET (or undocumented or current migraine that is severe, recurrent, requiring medication, and believed to be secondary to the ET; iii) a previous hemorrhage related to ET (or current hemorrhage related to ET); and iv) diabetes or hypertension requiring pharmacological therapy for greater than 60 months (past or present).

In a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented thrombosis (or undocumented or current thrombosis); iii) a previous hemorrhage related to ET (or current hemorrhage related to ET); and iv) diabetes or hypertension requiring pharmacological therapy for greater than 60 months (past or present).

Alternatively, as part of a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented thrombosis (or undocumented or current thrombosis); iii) a previous hemorrhage related to ET (or current hemorrhage related to ET); and iv) diabetes or hypertension requiring pharmacological therapy for greater than 60 months (past or present).

Alternatively, as part of a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented transient ischaemic attack (TIA); iii) a previous hemorrhage related to ET; and iv) diabetes or hypertension requiring pharmacological therapy for greater than 60 months.

Alternatively, as part of a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented transient ischaemic attack (TIA) (or undocumented or current TIA); iii) a previous hemorrhage related to ET (or current hemorrhage related to ET); and iv) diabetes or hypertension requiring pharmacological therapy for greater than 60 months (past or present).

Alternatively, as part of a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented erythromelalgia (or undocumented or current erythromelalgia); iii) a previous hemorrhage related to ET (or current hemorrhage related to ET); and iv) diabetes or hypertension requiring pharmacological therapy for greater than 60 months (past or present).

Alternatively, as part of a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented erythromelalgia (or undocumented or current erythromelalgia); iii) a previous hemorrhage related to ET (or current hemorrhage related to ET); and iv) diabetes or hypertension requiring pharmacological therapy for greater than 60 months (past or present).

Alternatively, as part of a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) the presence of JAK2, CALR or MPL mutation; and at least one additional characteristic selected from: i) the subject being age 60 or older; ii) a previous documented migraine that is severe, recurrent, requiring medication, and believed to be secondary to the ET (or undocumented or current migraine that is severe, recurrent, requiring medication, and believed to be secondary to the ET); iii) a previous hemorrhage related to ET (or current hemorrhage related to ET); and iv) diabetes or hypertension requiring pharmacological therapy for greater than 60 months (past or present).

Alternatively, as part of a fourth aspect, a subject to be treated by the methods described herein has high-risk ET characterized by the subject 1) having a platelet count greater than 1500×10⁹/L (at any point during the disease); 2) a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; 3) WHO criteria for BCR-ABL1+CML, PV, PMF, MDS, or other myeloid neoplasms not being met; 4) demonstration of another clonal marker (ASXL1, EZH2, TET2, IDH1/IDH2, SRSF2, or SR3B1 mutation) or no identifiable cause of thrombocytosis (e.g., infection, inflammation, iron deficiency anemia); and at least one additional characteristic selected from: i) the subject being age 60 or older; ii) a previous documented migraine that is severe, recurrent, requiring medication, and believed to be secondary to the ET (or undocumented or current migraine that is severe, recurrent, requiring medication, and believed to be secondary to the ET); iii) a previous hemorrhage related to ET (or current hemorrhage related to ET); and iv) diabetes or hypertension requiring pharmacological therapy for greater than 60 months (past or present).

In a fifth aspect, the subject's platelet count as defined herein (e.g., as in any one of the first through fourth aspects) is from 2 years or less, 1 year or less, at 11 months or less, 10 months or less, 9 months or less, 8 months or less, 7 months or less, 6 months or less, 5 months or less, four months or less, 3 months or less, two months or less, 1 month or less, 2 weeks or less, 1 week or less, 6 days or less, 5 days or less, 4 days or less, 2 days or less, or 1 day prior to the administration of 2-((4S)-6-(4-chlorophenyl)-1-methyl-4H-benzo[c]isoxazolo[4,5-e]azepin-4-yl)acetamide, or a pharmaceutically acceptable salt thereof.

The terms “subject” and “patient” may be used interchangeably, and refer to a human in need of treatment.

In a sixth aspect, a subject to be treated herein (e.g., as in any one of the first through fifth aspects) is intolerant, resistant, or has progressed relapsed to treatment with HU. As used herein, “intolerant” or “HU intolerance” refers to the subject's inability to tolerate the adverse effects of HU, generally at a therapeutic or sub-therapeutic dose. As used herein, “resistant” or “HU resistance” refers to reduction in effectiveness of HU within certain patients or patient population or refers to a subject who is unresponsive or demonstrates worsening of disease while on treatment with. In one aspect, intolerant or resistant to HU is as defined in Barosi et al., Leukemia. 2007; 21(2):277-280. A subject who is characterized as progressed/relapsed is one who at one time responded to treatment with HU, but who no longer responds.

Alternatively, as part of a sixth aspect, a subject to be treated herein (e.g., as in any one of the first through fifth aspects) is resistant to HU if one or more of the following criteria are met: 1) Platelet count >600×10⁹/L after 8 weeks of at least 2 g/day or MTD of HU (2.5 g/day in patients with a body weight>80 kg); 2) Platelet count >400×10⁹/L and WBC less than 25×10⁹/L at any dose of HU; 3) Platelet count >400×10⁹/L and Hb less than 10 g/dl at any dose of HU; 4) white blood cell count >15×10⁹/L; 5) progressive splenomegaly or hepatomegaly e.g., enlargement by more than 5 cm or appearance of new splenomegaly or hepatomegaly on HU treatment; 6) Not achieving the desired reduction of haematocrit or packed cell volume with the addition of HU in patients who do not tolerate frequent venesections after 8 weeks of at least 2 g/day of HU (2.5 g/day in patients with a body weight >80 kg); 7) Not achieving the desired stable reduction of WBC when leukocytes are a target of therapy after 8 weeks of at least 2 g/day or MTD of HU (2.5 g/day in patients with a body weight >80 kg; and/or 8) Cycling platelet counts on therapy.

Alternatively, as part of a sixth aspect, a subject to be treated herein (e.g., as in any one of the first through fifth aspects) is intolerant to HU if one or more of the following criteria are met: 1) Thrombosis or hemorrhage (including Transient Ischaemic Attack (TIA)) while on therapy; 2) Presence of leg ulcers or other unacceptable HU-related non-hematological toxicities, such as unacceptable mucocutaneous manifestations, gastrointestinal symptoms, pneumonitis or fever at any dose of HU; and/or 3) Disease-related symptoms not controlled by HU.

In a seventh aspect, a subject (e.g., as in any one of the first through sixth aspects) is excluded from disclosed treatment methods if the subject has met one or more of the following criteria: 1) uncontrolled rapid or paroxysmal atrial fibrillation, uncontrolled or unstable angina, recent (6 months) myocardial infarction or acute coronary syndrome or any clinically significant cardiac disease>NYHA Class II; 2) has myelofibrosis or has progressed to having myelofibrosis at any time during treatment; 3) has been previously administered a BET inhibitor other than Compound 1 or a pharmaceutically acceptable salt thereof; 4) inadequate liver function as defined by ALT/AST>1.5×ULN; and/or 5) inadequate renal function as defined by GFR<30 mls/min.

In an eighth aspect, a subject to be treated herein (e.g., as in any one of the first through seventh aspects) has not been previously administered another BET inhibitor prior to the administration of Compound 1 or a pharmaceutically acceptable salt thereof.

In a ninth aspect, Compound 1 in the methods described herein (e.g., as in any one of the first through eighth aspects) is administered as a crystalline form. Crystalline forms of Compound 1 are disclosed in U.S. Pat. No. 9,969,747. The entire contents of U.S. Pat. No. 9,969,747 which are incorporated by reference herein. Alternatively, as part of a ninth aspect, Compound 1 in the methods described herein (e.g., as in any one of the first through eighth aspects) is administered as a crystalline Form A. Alternatively, as part of a ninth aspect, Compound 1 in the methods described herein (e.g., as in any one of the first through eighth aspects), is administered as crystalline Form A characterized by at least three, at least four, at least five, or by six x-ray powder diffraction peaks at 20 angles selected from 4.73°, 18.09°, 18.48°, 18.80°, 19.70°, and 25.17°. Alternatively, as part of a ninth aspect, Compound 1 in the methods described herein (e.g., as in any one of the first through eighth aspects), is administered as crystalline Form A characterized by x-ray powder diffraction peaks at 20 angles 4.73°, 9.42°, 12.91°, 18.09°, 18.48°, 18.80°, 19.70°, 21.42°, and 25.17°. Alternatively, as part of a ninth aspect, Compound 1 in the methods described herein (e.g., as in any one of the first through eighth aspects), is administered as crystalline Form A characterized by x-ray powder diffraction peaks at 2Θ angles 4.73°, 8.110, 9.42°, 12.91°, 14.10°, 14.97°, 18.09°, 18.48°, 18.80°, 19.70°, 21.42°, and 25.17°, 26.07°, and 26.53°. Alternatively, as part of a ninth aspect, Compound 1 in the methods described herein (e.g., as in any one of the first through eighth aspects), is administered as hydrated (e.g., monohydrated) crystalline Form A characterized by at least three, at least four, at least five, or by six x-ray powder diffraction peaks at 2Θ angles selected from 4.73°, 18.09°, 18.48°, 18.80°, 19.70°, and 25.17°. Alternatively, as part of a ninth aspect, Compound 1 in the methods described herein (e.g., as in any one of the first through eighth aspects), is administered as hydrated (e.g., monohydrated) crystalline Form A characterized by x-ray powder diffraction peaks at 20 angles 4.73°, 9.42°, 12.91°, 18.09°, 18.48°, 18.80°, 19.70°, 21.42°, and 25.17°. Alternatively, as part of a ninth aspect, Compound 1 in the methods described herein (e.g., as in any one of the first through eighth aspects), is administered as hydrated (e.g., monohydrated) crystalline Form A characterized by x-ray powder diffraction peaks at 20 angles 4.73°, 8.110, 9.42°, 12.91°, 14.10°, 14.97°, 18.09°, 18.48°, 18.80°, 19.70°, 21.42°, and 25.17°, 26.07°, and 26.530.

The terms “treatment,” “treat,” and “treating” refer to reversing, alleviating, reducing the likelihood of developing, or inhibiting the progress of thrombocythemia (e.g., ET and/or high risk ET), or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to prevent or delay their recurrence. Symptoms of ET include, but are not limited to, increased production of megakaryocytes, blood clots, enlargement of the spleen, bleeding in several parts of the body and/or clotting episodes such as strokes, pain in the legs and difficulty breathing, weakness, headaches, or a burning, tingling or prickling sensation in the skin, dizziness, nosebleeds, easy bruising, bleeding from the mouth or gums, bloody stool and/or or anal bleeding due to bleeding in the intestines.

The term “effective amount” or “therapeutically effective amount” are used interchangeably and include an amount of a compound described herein that will elicit a desired medical response in a subject having thrombocythemia (e.g., ET and/or high risk ET), e.g., reducing the symptoms of and/or slowing the progression of the disease.

In a tenth aspect, a subject treated herein (e.g., as in any one of the first through ninth aspects) is said to have a complete hematological response (CHR) to treatment of Compound 1, or a pharmaceutically acceptable salt thereof, if after the timeframe from 1 month to 6 months of treatment, the subject has a platelet count of ≤400×10⁹/L for at least one month, a hemoglobin level of >10 g/dL, and white blood cell count of →10×10⁹/L, and a myeloproliferative neoplasm symptom assessment form (MPN-SAF) Total Symptom Score (TSS) Response, defined as an at least 50% reduction in MPN-SAF TSS from baseline, at Week 24. See e.g., J Clin Oncol. 2012 Nov. 20; 30(33): 4098-4103. Alternatively, as part of a tenth aspect, a subject treated herein (e.g., as in any one of the first through ninth aspects) is said to have a complete hematological response (CHR) to treatment of Compound 1, or a pharmaceutically acceptable salt thereof, if after the timeframe from 1 month to 6 months of treatment, the subject has a platelet count of ≤400×10⁹/L for at least one month, a white blood cell count of ≤10×10⁹/L, normal spleen size, and no disease-related symptoms.

In an eleventh aspect, a subject treated herein (e.g., as in any one of the first through ninth aspects) a subject is said to have a partial hematological response (CHR) to treatment of Compound 1, or a pharmaceutically acceptable salt thereof, if after the timeframe from 1 month to 6 months of treatment, the subject has a platelet count ≤600×10⁹/L, a platelet count decrease of >50% from baseline, or a platelet count of <400×10⁹/L with anemia or leukocytosis.

In a twelfth aspect, the subject treated by the methods described herein (e.g., as in any one of the first through eleventh aspects) is also characterized as being anemic. As part of a twelfth aspect, a subject of the present disclosure is characterized as anemic if their hemoglobin value is less than 13.5 g/dL of blood for a male subject or less than 12.0 g/dL of blood for a female subject. As part of a twelfth aspect, the subject treated by the methods described herein (e.g., as in any one of the first through eleventh aspects) is characterized as being anemic if their hemoglobin value is less than 10.0 g/dL. As part of a twelfth aspect, subjects treated by the present methods (e.g., as in any one of the first through eleventh aspects) therefore include those having hemoglobin values less than 13.0 g/dL, less than 12.5 g/dL, less than 12.0 g/dL, less than 11.5 g/dL, less than 11.0 g/dL, less than 10.5 g/dL, less than 10.0 g/dL, less than 9.5 g/dL, less than 9.0 g/dL, or less than 8.5 g/dL for male subjects and less than 11.5 g/dL, less than 11.0 g/dL, less than 10.5 g/dL, less than 10.0 g/dL, less than 9.5 g/dL, less than 9.0 g/dL, or less than 8.5 g/dL for female subjects. Alternatively, as part of a twelfth aspect, a subject (e.g., as in any one of the first through eleventh aspects) is characterized as being anemic if their hemoglobin value ranges from 7.5 g/dL of blood to 13.5 g/dL of blood for a male subject or from 7.5 g/dL of blood to 12.0 g/dL of blood for a female subject. Alternatively, as part of a twelfth aspect, a subject (e.g., as in any one of the first through eleventh aspects) is characterized as being anemic if their hemoglobin value ranges from 7.5 g/dL of blood to 10.5 g/dL of blood for a male subject or from 7.5 g/dL of blood to 10.5 g/dL of blood for a female subject. Alternatively, as part of a twelfth aspect, a subject (e.g., as in any one of the first through eleventh aspects) is characterized as being anemic if their hemoglobin value ranges from 7.5 g/dL of blood to 10.0 g/dL of blood for a male subject or from 7.5 g/dL of blood to 10.0 g/dL of blood for a female subject. Alternatively, as part of a twelfth aspect, a subject (e.g., as in any one of the first through eleventh aspects) is characterized as being anemic if their hemoglobin value ranges from 7.7 g/dL of blood to 10.7 g/dL of blood for a male subject or from 7.7 g/dL of blood to 10.5 g/dL of blood for a female subject. Alternatively, as part of a twelfth aspect, a subject (e.g., as in any one of the first through eleventh aspects) is characterized as being anemic if their hemoglobin value ranges from 7.7 g/dL of blood to 10.0 g/dL of blood for a male subject or from 7.7 g/dL of blood to 10.0 g/dL of blood for a female subject.

In a thirteenth aspect, the subject treated by the methods described herein (e.g., as in any one of the first through twelfth aspects) is further characterized as being leukopenic. As part of a thirteenth aspect, a subject (e.g., as in any one of the first through twelfth aspects) is characterized to be leukopenic if their white blood cell (WBC) count is less than 4,000 WBCs/μL of blood. Alternatively, as part of a thirteenth aspect, subjects treated by the present methods (e.g., as in any one of the first through twelfth aspects) include those having WBC counts of less than 3,500 WBCs/μL, 3,200 WBCs/μL, 3,000 WBCs/μL, or 2,500 WBCs/μL.

In a fourteenth aspect, the subject treated by the methods described herein (e.g., as in any one of the first through thirteenth aspect) is further characterized as also being neutropenic. As part of a fourteenth aspect, the subject treated by the methods described herein is (e.g., as in any one of the first through thirteenth aspects) is characterized as neutropenic if their neutrophil count is less than 1500 neutrophils/μL of blood. Alternatively, as part of a fourteenth aspect, subjects treated by the present methods (e.g., as in any one of the first through thirteenth aspects) include those having neutrophil counts of less than 1250 neutrophils/μL, 1000 neutrophils/μL, 750 neutrophils/μL, or 500 neutrophils/μL.

In a fifteenth aspect, subjects treated by the present methods (e.g., as in any one of the first through fourteenth aspects) are transfusion dependent prior to treatment with Compound 1. In some aspects, “transfusion dependent” means that a subject requires red blood cell (RBC) transfusions in order to maintain an acceptable level of hemoglobin. An acceptable level of hemoglobin is determined by those skill in the art and can range from e.g., from 13.5 to 17.5 g/dL of blood for men and from 12.0 to 15.5 g/dL of blood in women.

In a sixteenth aspect, subjects of the present methods (e.g., as in any one of the first through fifteenth aspects) also have an enlarged spleen prior to treatment. As part of a sixteenth aspect, subjects treated by the present methods experience a reduction in spleen size. As part of a sixteenth aspect, the reduction in spleen size comprises a 10% or more (e.g., a 15% or more, a 20% or more, a 25% or more, a 30% or more, a 35% or more, a 40% or more, a 45% or more, a 50% or more, a 55% or more, a 60% or more, or a 65% or more reduction in spleen volume from baseline. Alternatively, as part of a sixteenth aspect, the reduction comprises from a 10% to a 65% reduction in spleen volume from baseline.

In a seventeenth aspect, subjects of the present methods (e.g., as in any one of the first through sixteenth aspects) comprise a MPN-SAF score of greater than 10.

In an eighteenth aspect, subjects of the present methods (e.g., as in any one of the first through seventeenth aspects) are transfusion dependent.

In a nineteenth aspect, subjects treated by the present methods (e.g., as in any one of the first through eighteenth aspects) have a reduction in the number of blood transfusions.

In a twentieth aspect, subjects treated by the present methods (e.g., as in any one of the first through nineteenth aspects) experience a reduction in headaches.

In a twenty-first aspect, subjects treated by the present methods (e.g., as in any one of the first through twentieth aspects) experience an increase in hemoglobin values.

In a twenty-second aspect, subjects treated by the present methods (e.g., as in any one of the first through twenty-first aspects) experience an improvement in bone marrow fibrosis as determined e.g., by the bone marrow fibrosis grading scale (see Thiele J et al., Haematologica, 2005, 90, 1128). In one aspect, an improvement is defined as at least one grade improvement in the bone marrow fibrosis/reticulin grading compared to baseline.

In a twenty-third aspect, subjects treated by the present methods (e.g., as in any one of the first through twenty-second aspects) experience a reduction in pro-inflammatory cytokines such as e.g., CRP, IL-8, and/or IL-18.

Compound 1 can be formulated as a pharmaceutical composition and administered to the subject in a variety of forms adapted to the chosen route of administration. Typical routes of administering such pharmaceutical compositions include, without limitation, oral, topical, buccal, transdermal, inhalation, parenteral, sublingual, rectal, vaginal, and intranasal. The term parenteral as used herein includes subcutaneous injections, intravenous, intramuscular, intrathecal, intrasternal injection or infusion techniques. Methods of formulating pharmaceutical compositions are well known in the art, for example, as disclosed in “Remington: The Science and Practice of Pharmacy,” University of the Sciences in Philadelphia, ed., 21st edition, 2005, Lippincott, Williams & Wilkins, Philadelphia, PA.

Pharmaceutical compositions can be prepared by combining a compound of the methods described herein with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Thus, Compound 1, or a pharmaceutically acceptable salt thereof, may be systemically administered, e.g., orally, in combination with a pharmaceutically acceptable excipient such as an inert diluent or an assimilable edible carrier. Compound 1, or a pharmaceutically acceptable salt thereof, may be enclosed in hard or soft shell gelatin capsules, may be compressed into tablets or may be incorporated directly with the food of the patient's diet. For oral therapeutic administration, Compound 1, or a pharmaceutically acceptable salt thereof, may be combined with one or more excipients and used in the form of ingestible tablets, buccal tablets, troches, capsules, elixirs, suspensions, syrups, wafers, and the like.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of Compound 1, or a pharmaceutically acceptable salt thereof, described herein in the composition will also depend upon the particular compound in the composition. In one aspect, Compound 1, or a pharmaceutically acceptable salt thereof, may be formulated at a dose of from 50 mg to 500 mg for e.g., administration once, twice, or three times daily. For example, Compound 1 may be administered at a dosage of from 50 mg to 300 mg/day, from 75 mg to 300 mg/day, from 100 mg to 300 mg/day, from 100 mg to 200 mg/day, from 150 mg to 250 mg/day, or at 100 mg/day, 125 mg/day, 150 mg/day, 175 mg/day, 200 mg/day, 225 mg/day, or 250 mg/day.

### Exemplification

Compound 1 can be obtained following the procedures described in U.S. Pat. No. 8,796,261 and WO 2015/195862. Crystalline forms can be obtained following the procedures described in U.S. Pat. No. 9,969,747.

### Inhibitory Effect on Cytokine Release In Vitro

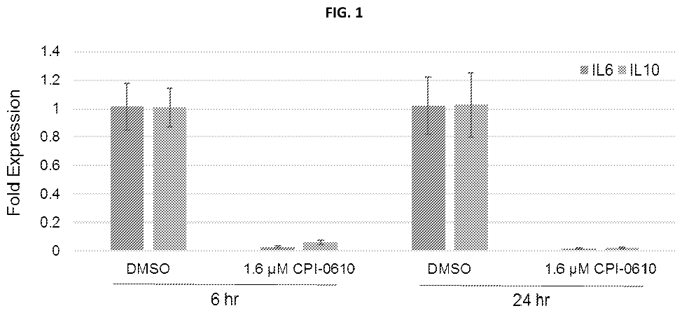

Compound 1 was assessed for its ability to suppress the expression of NF-κB target genes in two experiments. In one experiment, THP-1 acute leukemia cell lines were exposed to lipopolysaccharide treatment and then Compound 1 for 16 hours. IL6 release from the THP-1 acute leukemia cells was inhibited, with an IC₅₀ of 0.069 μM. In the other experiment, the ability of Compound 1 to suppress both IL6 and IL10 expression in TMD8 ABC-DLBCL cells was investigated (data on file). TMD8 cells were incubated with DMSO or 1.6 μM Compound 1 for 6 or 24 hours. RNA was then extracted from the cells and quantified using qRT-PCR. As shown in FIG. 1, Compound 1 substantially suppressed mRNA transcription of both IL6 and IL10 after 6 and 24 hours of treatment.

### Effect of Compound 1 as a Single Agent on Megakaryocyte Differentiation

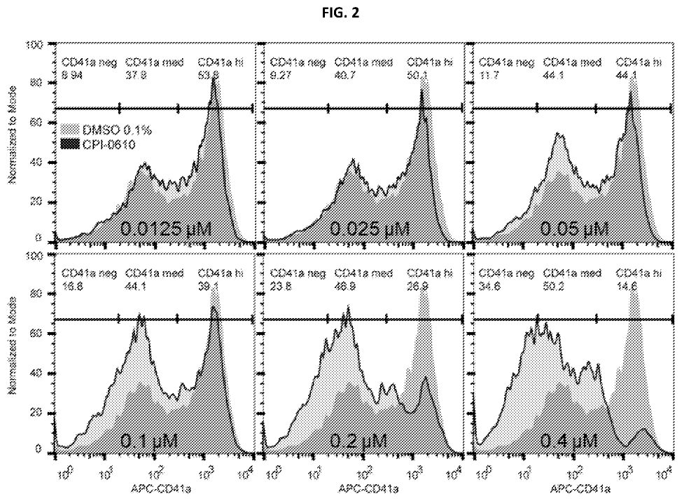

The effects of Compound 1 on megakaryocyte differentiation and proliferation were evaluated using CD34+ cells isolated from healthy donor bone marrow (data on file). The CD34+ cells were grown in megakaryocyte differentiation serum-free stem cell differentiation base medium with a megakaryocyte-driving cytokine cocktail for 14 days with DMSO or Compound 1 at concentrations ranging from 3 nM to 500 nM. The cells were then stained for CD34 (progenitor marker), CD45 (leukocyte marker) and CD41a (mature megakaryocyte marker) and assessed by FACS for viability and marker expression. CD41a expression and cell size were used as markers of megakaryocyte differentiation. Compound 1 reduced the number of cells with high CD41a expression in a concentration-dependent manner. The shift from high to low CD41a expression began at approximately 50 nM, with pronounced effects observed at 200 to 500 nM, as shown in FIG. 2. The loss of CD41a-high-expressing cells suggests impaired megakaryocyte differentiation and loss of mature megakaryocytes.

Aberrant megakaryocytes (Mk) are drivers of inflammation and altered hematopoiesis in ET. Through its inhibitory effects on Mk differentiation and proliferation, treatment with Compound 1 should diminish the quantity of platelets and the pro-inflammatory cytokines that are released from megakaryocytes and may reduce the mutant allele burden.

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating essential thrombocythemia (ET) in a subject in need thereof, comprising administering to the subject a therapeutically effective amount of 2-((4S)-6-(4-chlorophenyl)-1-methyl-4H-benzo[c]isoxazolo[4,5-e]azepin-4-yl)acetamide, or a pharmaceutically acceptable salt thereof.

2. The method of claim 1, wherein the ET is characterized by the subject having
a platelet count of greater than or equal to 450×10⁹/L,
a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; and
the presence of JAK2, CALR or MPL mutation, and wherein the WHO criteria for BCR-ABL1+CML, PV, PMF, MDS, or other myeloid neoplasms are not met.

3. The method of claim 1, wherein the ET is characterized by the subject having
a platelet count of greater than or equal to 450×10⁹/L,
a bone marrow biopsy showing proliferation mainly of the megakaryocyte lineage with increased numbers of enlarged, mature megakaryocytes with hyperlobulated nuclei and no significant increase or left shift in neutrophil granulopoiesis or erythropoiesis and very rarely minor (grade 1) increase in reticulin fibers; and
demonstration of another clonal marker or no identifiable cause of thrombocytosis, and wherein the WHO criteria for BCR-ABL1+CML, PV, PMF, MDS, or other myeloid neoplasms are not met.

4. The method of claim 3, wherein the clonal marker is selected from an ASXL1, EZH2, TET2, IDH1/IDH2, SRSF2, or SR3B1 mutation.

5. The method of claim 3, wherein the cause of thrombocytosis is selected from an infection, inflammation, iron deficiency, and anemia.

6. The method of claim 1, wherein the ET is characterized as high-risk ET.

7. The method of claim 1, wherein the subject is age 60 or older, had or has a thrombosis, had or has a hemorrhage related to ET, and had or has diabetes or hypertension requiring pharmacological therapy for greater than 60 months; or wherein the subject is age 60 or older, had or has a transient ischaemic attack (TIA), had or has a hemorrhage related to ET, and had or has diabetes or hypertension requiring pharmacological therapy for greater than 60 month; or wherein the subject is age 60 or older, had or has a erythromelalgia, had or has a hemorrhage related to ET, and had or has diabetes or hypertension requiring pharmacological therapy for greater than 60 months; or wherein the subject is age 60 or older had or has a migraine that is severe, recurrent, requiring medication; had or has a hemorrhage related to ET, and had or has diabetes or hypertension requiring pharmacological therapy for greater than 60 months.

8-10. (canceled)

11. The method of claim 1, wherein the subject has a platelet count greater than 1500×10⁹/L, is age 60 or older, had or has a thrombosis, had or has a hemorrhage related to ET; and had or has diabetes or hypertension requiring pharmacological therapy for greater than 60 months; or wherein the subject has a platelet count greater than 1500×10⁹/L, is age 60 or older, had or has a transient ischaemic attack (TIA), had or has a hemorrhage related to ET; and had or has diabetes or hypertension requiring pharmacological therapy for greater than 60 months; or wherein the subject has a platelet count greater than 1500×10⁹/L, is age 60 or older, had or has a erythromelalgia, had or has a hemorrhage related to ET; and had or has diabetes or hypertension requiring pharmacological therapy for greater than 60 months; or wherein the subject has a platelet count greater than 1500×10⁹/L; is age 60 or older: had or has a migraine that is severe, recurrent, requiring medication; had or has a hemorrhage related to ET; and had or has diabetes or hypertension requiring pharmacological therapy for greater than 60 months.

12-14. (canceled)

15. The method of claim 1, wherein the subject was previously administered hydroxyurea.

16. The method of claim 1, wherein the subject is resistant to hydroxyurea.

17. The method of claim 1, wherein the subject is intolerant to hydroxyurea.

18. The method of claim 1, wherein the subject is anemic.

19. The method of claim 1, wherein the subject has a hemoglobin count of less than 10 g/dL.

20. The method of claim 1, wherein the subject has an enlarged spleen or liver.

21. The method of claim 1, wherein the subject is neutropenic.

22. The method of claim 1, wherein the subject's absolute neutrophil count is less than 1000 neutrophils/μL of blood.

23. The method of claim 1, wherein the subject is administered from 100 mg/day to 300 mg/day of 2-((4S)-6-(4-chlorophenyl)-1-methyl-4H-benzo[c]isoxazolo[4,5-e]azepin-4-yl)acetamide.

24. The method of claim 1, wherein the 2-((4S)-6-(4-chlorophenyl)-1-methyl-4H-benzo[c]isoxazolo[4,5-e]azepin-4-yl)acetamide is a monohydrate.

25. The method of claim 1, wherein the 2-((4S)-6-(4-chlorophenyl)-1-methyl-4H-benzo[c]isoxazolo[4,5-e]azepin-4-yl)acetamide is crystalline Form A monohydrate.
